(19) United States
(12) Patent Application Publication
(10) Pub. No.: US 2025/0115847 A1
(43) Pub. Date: Apr. 10, 2025

(54) CELL PRODUCTION METHOD, CULTURE VESSEL, AND DEVICE FOR OSCILLATING CULTURE

(71) Applicants: THE UNIVERSITY OF TOKYO, Tokyo (JP); ABLE Corporation, Tokyo (JP)

(72) Inventors: Takashi OKADA, Tokyo (JP); Yuji TSUNEKAWA, Tokyo (JP); Shutaro ISHIKAWA, Tokyo (JP)

(21) Appl. No.: 18/832,068

(22) PCT Filed: Jan. 20, 2023
(86) PCT No.: PCT/JP2023/001673

§ 371 Date: Jul. 22, 2024

(30) Foreign Application Priority Data
Feb. 3, 2022 (JP) 2022-015569

(51) Int. Cl. C12M 3/06; C12M 1/00; C12M 1/34; C12M 3/00

(52) U.S. Cl. CPC C12M 27/16; C12M 23/14; C12M 23/46; C12M 41/34

(57) ABSTRACT

Provision of a cell production method, in particular, a method in which carriers are suspended and oscillated in a culture fluid, which is capable of reducing physical stress imposed on animal cells or a transfection reagent and improving cell culture efficiency or transfection efficiency.

The cell production method includes a culturing step for culturing cells in a culture vessel 2, which holds at least cells, a culture fluid, and a carrier C while the vessel is oscillated on an oscillation plate 102, in which the oscillation plate 102 is tilted only in one direction with respect to a horizontal state.

### TECHNICAL FIELD

The present invention relates to a cell production method and a culture vessel used therefor, and an oscillating-type culture device.

### BACKGROUND ART

In culturing cells in a liquid medium (culture fluid), a certain degree of agitation of the culture fluid is necessary to ensure that substances necessary for cell culture are properly supplied to the cells.

Patent Document 1 describes a conventional technique for the process of transfection of cells attached to a carrier in a liquid medium. As described in Patent Document 1, after a transfection reagent is added, a certain degree of agitation is still required to properly contact the cells with a sufficient amount of the transfection reagent.

One of the methods used to agitate such a liquid is the oscillating method, in which a culture vessel (generally a bag-type culture vessel) is rocked from side to side, and the liquid in the culture vessel is agitated by moving the liquid back and forth from side to side. The oscillating method is a method with relatively low physical stress on cells. A conventional technique for such an oscillating method is described in Patent Document 2.

In many of the culture vessels used in the oscillating method, flexible culture bags made of resin are used and are made to be disposable, eliminating the need for cleaning and sterilizing the culture vessels and preventing occurrence of contamination.

Conventional techniques for such culture vessels (culture bags) are disclosed in Patent Documents 3 to 5.

### CITATION LIST

### Patent Literature

Patent Document 1: International Publication WO 2019/189545
Patent Document 2: U.S. Pat. No. 6,190,913
Patent Document 3: Japanese Patent Application Publication 2019-135968
Patent Document 4: Japan Patent No. 5214714
Patent Document 5: Japanese Patent Application Publication 2021-038000

### SUMMARY OF INVENTION

### Technical Problem

Although the oscillating method imparts relatively less physical stress on cells as mentioned above, some cell types are less resistant to physical stress, and transfection reagents are also very sensitive to physical stress. Therefore, there was a problem with the conventional oscillating method of causing undesirable physical stress on cells or transfection reagents. In other words, as the liquid in the culture vessel moves back and forth from side to side, carriers also move back and forth from side to side, causing the cells to be subjected to physical stress caused by collision of cells attached to each carrier.

In view of the above points, it is an object of the present invention to provide a cell production method, particularly, a method of suspending and oscillating carriers in a culture fluid, which can reduce physical stress on cells or transfection reagents.

### Solution to Problem

### (Configuration 1)

A cell production method in which, in a culturing step for culturing cells while oscillating a culture vessel containing at least cells, a culture fluid, and a carrier on an oscillating plate, tilting the oscillating plate only in one direction with respect to a horizontal state.

### (Configuration 2)

The cell production method according to Configuration 1, in which the culturing step includes a pre-culturing step and a main culturing step.

### (Configuration 3)

The cell production method according to Configuration 1 or 2, including a transfection step in which, after the culturing step, a transfection is performed while oscillating the culture vessel containing at least a transfection reagent and the carrier having cells attached thereto on the oscillating plate, such that the oscillating plate is tilted only in one direction with respect to a horizontal state.

### (Configuration 4)

The cell production method according to Configurations 1 to 3, in which a tilt angle of the oscillating plate during oscillation is within a range between 0° and 45° with respect to a horizontal plane.

### (Configuration 5)

The cell production method according to any one of Configurations 1 to 4, in which a part of the culture fluid is moved from the culture vessel to a measuring tank to measure dissolved oxygen or pH of the culture fluid in the measuring tank, and delivery of the culture fluid between the culture vessel and the measuring tank is carried out by controlling an internal pressure of the measuring tank.

### (Configuration 6)

The culture vessel used in the cell production method according to any one of Configurations 1 to 5, including a bag portion formed by joining end regions of opposing sheets leaving an opening, and a plate member having a port member and occluding the opening.

### (Configuration 7)

The culture vessel according to Configuration 6, including a fastening member for closely attaching the plate member to the opening.

### (Configuration 8)

The culture vessel according to Configuration 7, in which the plate member is circular, and in which the fastening member includes two semi-circular members that are substantially circular by being connected to each other, a flange portion formed on one end of the semi-circular members, and a fastening force applying member that generates a fastening force for closely attaching the plate member to the opening.

### (Configuration 9)

The culture vessel according to Configuration 8, in which the opposing sheets are formed by folding a single sheet, and a joined portion of a folded sheet is sandwiched by the flange portions.

### (Configuration 10)

The culture vessel according to any one of Configurations 6 to 9, including a sealing member on an outer periphery of the plate member.

### (Configuration 11)

The culture vessel according to any one of Configurations 6 to 10, further including an attachable portion for attachment to the oscillating plate.

### (Configuration 12)

An oscillating-type culture device used in the cell production method according to any one of Configurations 1 to 5, including the oscillating plate, an oscillation control unit for controlling oscillation of the oscillating plate for tilting the oscillating plate only in one direction with respect to a horizontal state, a sensor value acquisition unit for acquiring a sensor value from a sensor attached to the culture vessel, and a gas supply control unit for supplying oxygen or carbon dioxide into the culture vessel based on the sensor value.

### (Configuration 13)

A culture vessel for cell culture, including a bag portion formed by joining end regions of opposing sheets leaving an opening, and a plate member having a port member and occluding the opening.

### Advantageous Effects of Invention

According to the cell production method of the present invention, it is possible to reduce physical stress on cells or transfection reagents.

### BRIEF DESCRIPTION OF DRAWINGS

FIG. 1 is a block diagram schematically showing a configuration of a culture device for use in a cell production method of Embodiment 1 of the present invention.

FIG. 2 is an explanatory view explaining the cell production method of Embodiment 1.

FIG. 3 is a flowchart schematically showing a process of the cell production method of Embodiment 1.

FIG. 4 shows a culture vessel of Embodiment 2.

FIG. 5 explains a bag portion of Embodiment 2.

FIG. 6 shows a plate member of Embodiment 2.

FIG. 7 shows a fastening member of Embodiment 2.

### DESCRIPTION OF EMBODIMENTS

Embodiments of the present invention are specifically explained below referring to the drawings. The embodiments given below are some of the embodiments in embodying the present invention, and by no means limit the present invention within such a scope.

### Embodiment 1

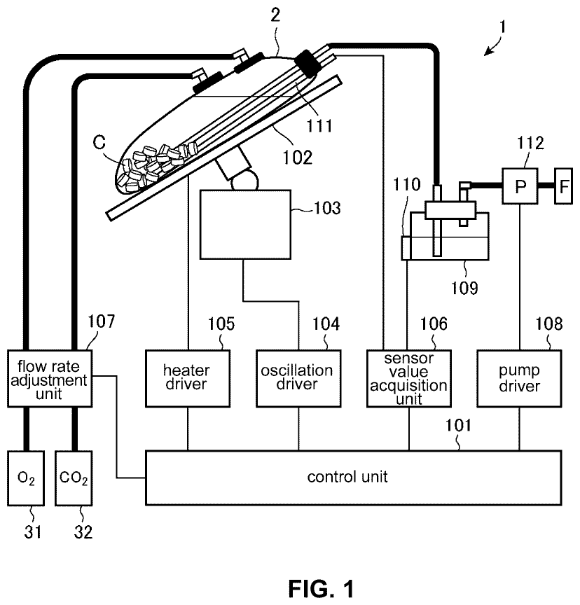

FIG. 1 is a block diagram schematically showing a configuration of a culture device used for a cell production method of an embodiment of the present invention.

An oscillating-type culture device 1 includes:
an oscillating plate 102 on which a culture vessel 2 is installed and having a heater;
an oscillating motion unit 103 for oscillating the oscillating plate 102 such as by including a motor;
an oscillation driver 104 for driving the oscillating motion unit 103;
a heater driver 105 for supplying power to the heater of the oscillating plate 102;
a measuring tank 109;
a sensor (dissolved oxygen sensor, pH sensor) 110 attached to the measuring tank 109;
a temperature sensor 111 attached to the culture vessel 2;
a sensor value acquisition unit 106 to acquire a sensor value (perform A/D conversion, etc.) from each sensor (temperature sensor, dissolved oxygen sensor, pH sensor);
a pump 112 for delivery of a culture fluid, etc. between the culture vessel 2 and the measuring tank 109;
a pump driver 108 for driving the pump 112;
a flow rate adjustment unit 107 for adjusting a supply amount of oxygen and carbon dioxide to the culture vessel 2 from an oxygen cylinder 31 and a carbon dioxide cylinder 32 via a delivery tube or pipe connected to a port of the culture vessel 2; and
a control unit 101 for controlling each part.

Although not shown, the oscillating-type culture device 1 further includes an output portion and an input portion as user interface.

The oscillating plate 102 oscillates at a set speed (rotational speed/angular speed), cycle, and angle by the oscillating motion unit 103 being controlled by the control unit 101. The operating conditions of oscillation (speed, cycle, angle, etc.) may, for example, be selected by a user from those set in the device in advance, or may be set by a user on a case-by-case basis.

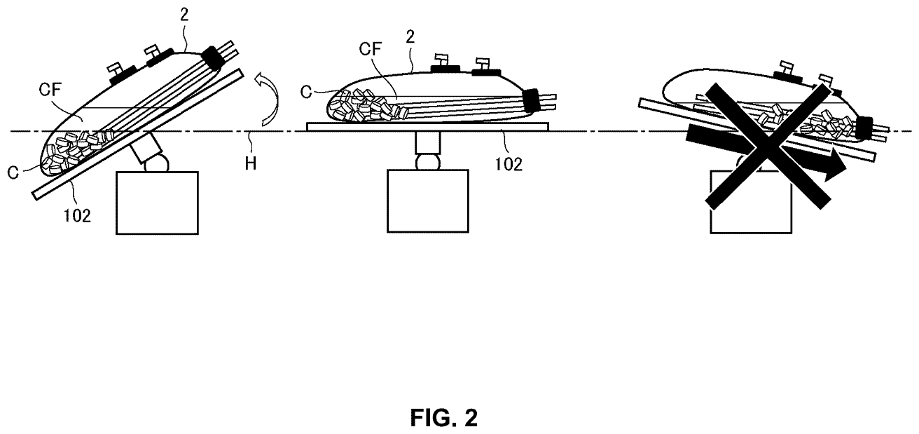

In the cell production method of this embodiment, the culture fluid, etc. is agitated by tilting the oscillating plate 102 only in one direction with respect to a horizontal state. “Tilting only in one direction with respect to a horizontal state” means that in the oscillating motion, once tilted in a certain direction with respect to a horizontal plane, it is not tilted beyond the horizontal plane in the opposite direction. In other words, since this is an “oscillating motion”, a motion to tilt in one direction is naturally followed by tilting in the opposite direction; however, the angle of the oscillating plate 102 during the oscillating motion is not changed across the horizontal plane. In other words, this embodiment repeats the driving in which the oscillating plate 102 is rotated (changes its posture) the tilted state toward the horizontal state and the rotational motion is reversed upon reaching the horizontal state as shown in the center of FIG. 2, and the driving in which the oscillating plate 102 is rotated from the horizontal state toward the tilted state as shown in the right side of FIG. 2 and the rotational motion is reversed when the oscillating plate 102 reaches a predetermined angle.

The measuring tank 109 includes a sensor (dissolved oxygen sensor, pH sensor) 110, in which a part of the culture fluid, etc. is moved from the culture vessel 2 to the measuring tank 109 to measure dissolved oxygen or pH of the culture fluid, etc. The measuring tank 109 of this embodiment further includes a temperature sensor. Although not shown, the measuring tank 109 is also provided with a heater, and the temperature is controlled by the heater driver and the control unit.

In oscillating-type culture devices, culture bags (flexible containers) made of resin sheets are often used as culture vessels, and the shape of the culture vessel is not stable because of the oscillating motion of the culture bag. It is difficult to use an optical sensor or the like for such unstable vessels, and stable measurement is difficult. Therefore, a part of the culture fluid, etc. is moved from the culture vessel 2 to the measuring tank 109 to enable stable measurement in the measuring tank 109.

The measuring tank 109 of this embodiment is provided with an agitation device (not shown). The agitation device of the measuring tank 109 of this embodiment is of a type in which an agitating member provided with a magnet in the tank is moved by magnetic force from the outside. The agitation device can be of any configuration, and the measuring tank may not be provided with an agitation device (since an agitating action can be obtained by circulation by a pump, which will be explained below).

Delivery of the culture fluid, etc. between the culture vessel 2 and the measuring tank 109 is carried out by controlling the internal pressure in the measuring tank 109.

Increasing or decreasing the internal pressure of the measuring tank 109 is performed by the pump 112 and the pump driver 108 that drives the pump 112. A decrease in the internal pressure of the measuring tank 109 causes the culture fluid, etc. to be delivered from the culture vessel 2 to the measuring tank 109, and an increase in the internal pressure of the measuring tank 109 causes the culture fluid, etc. to be delivered from the measuring tank 109 to the culture vessel 2. In this embodiment, liquid level sensor (not shown) is provided in the measuring tank 109, and based on a sensor value of the liquid level sensor, the control unit 101 and the pump driver 108 carry out the operation to switch the operation of the pump to increase the internal pressure of the measuring tank 109 when the liquid level in the measuring tank 109 rises to a predetermined water level (first water level), and to switch the operation of the pump to decrease the internal pressure of the measuring tank 109 when the liquid level in the measuring tank 109 falls to a predetermined water level (second water level).

By repeating this operation, in the culturing step and the transfection step, the liquid in the culture vessel 2 and the liquid in the measuring tank 109 are kept in almost the same state by the liquid being constantly circulated between the culture vessel 2 and the measuring tank 109 and the temperature being controlled as mentioned above (Note that the temperature control (and configuration therefor) in the measuring tank 109 is not always necessary depending on the configuration of the device, such as a short liquid delivery path and frequent circulation of the liquid, or installation condition of the device, such as being installed in a stable temperature environment).

In performing this type of liquid delivery, a peristaltic pump is often disposed in the liquid delivery path (in this case, between the culture vessel 2 and the measuring tank 109). However, disposing the peristaltic pump in the liquid delivery path causes physical stress on the liquid, which is undesirable for transfection reagents, as they are sensitive to physical stress. In contrast, the oscillating-type culture device 1 of this embodiment has an excellent functional effect of reducing the physical stress on the liquid during delivery due to the above configuration.

Any type of pump can be used as the pump 112. In this embodiment, a peristaltic pump is used and is connected to the atmosphere via a filter F.

The control unit 101 controls the oscillating motion of the oscillating plate 102 by controlling the oscillation driver 104. Further, the culture vessel 2 is controlled to be kept at an appropriate temperature by performing feedback control based on the measured value of the temperature sensor 111 to the heater driver 105 (similarly, the temperature of the measuring tank 109 is also controlled). Feedback control based on the measured values of the sensors (dissolved oxygen sensor, pH sensor) 110 in the measuring tank 109 is performed to the flow rate adjustment unit 107 to maintain an appropriate amount of oxygen and pH of the culture fluid, etc. in the culture vessel 2 (feedback control of the culture fluid, etc. in the culture vessel 2 is performed based on the measured values in the measuring tank 109).

The control unit 101 and the oscillation driver 104 constitute “an oscillation control unit that controls to tilt the oscillation of the oscillating plate only in one direction with respect to a horizontal state”. The control unit 101, the sensor value acquisition unit 106, and the flow rate adjustment unit 107 constitute “a gas supply control unit for supplying oxygen or carbon dioxide into the culture vessel based on the sensor value of the measuring tank”. The control unit 101 and the pump driver 108 constitute “a liquid delivery control unit that delivers liquid between the culture vessel and the measuring tank by controlling the internal pressure of the measuring tank”.

In this embodiment, the culture vessel 2 is a disposable culture bag made of a synthetic resin film, provided with various ports (connections for connecting tubes and pipes for supplying/discharging liquid or gas to/from the culture vessel, attaching sections for attaching a temperature sensor, sampling ports for taking samples from inside the culture vessel, etc.).

During the culturing step of cells, at least the cells, the culture fluid, and the carrier C are contained in the culture vessel 2, and during the transfection step, at least the transfection reagent and the carrier C with cultured cells attached thereto are contained in the culture vessel 2. HEK293 cells can be exemplified as cells applicable to this embodiment, but the type of cells is not limited thereto. An appropriate amount of fabric carrier can be used as the carrier, but the shape and material of the carrier are not limited thereto.

Furthermore, although this embodiment uses a culture bag made of a synthetic resin film as an example of the culture vessel, the invention is not limited thereto, but any culture vessel that can be placed on the oscillating plate 102 can be used.

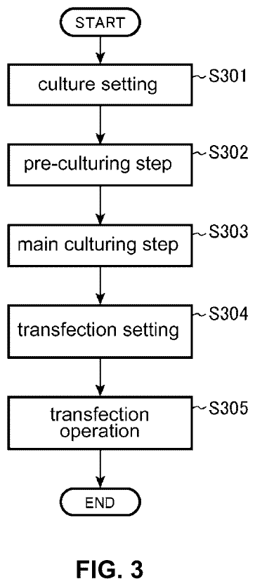

Next, an outline of the process of the cell production method of this embodiment is described with reference to FIG. 3.

In Step 301, a setting is made for cell culture. In this setting, the culture vessel 2 containing the culture fluid, the carrier C, and adhesive cells is installed (with various connections) to the oscillating-type culture device 1, and the culture fluid is also placed in the measuring tank 109.

Once the setting for cell culture is done, the culturing step begins. The culturing step includes two stages: a pre-culturing step and a main culturing step. The reason for the two-stage culturing is for the purpose of use when a cell suspension in a specific culturing phase is sought to be prepared at a specific time. Specifically, in the pre-culturing step, the cells are cultured in a small amount of medium up to a stationary phase, and then the cells are cultured in the main culturing step for a certain period of time with uniform cell concentration. In this way, as long as the culture conditions arranged, a cell suspension of approximately the same concentration can be obtained each time at a specific time.

The first step in the culturing step is the pre-culturing step (Step 302). In the motion of the pre-culturing step, agitation (oscillation) of the culture fluid is reduced in order to reduce generation of physical stress until the cells adhere to the carrier C. For example, an operation mode in which oscillating motion (frequency) is about once (one reciprocation) per hour is performed for about 6 hours. In the operation mode of pre-culturing in this embodiment, only the temperature is controlled, and oxygen amount and pH (supply of oxygen and carbon dioxide) are not controlled (pump 112 is not operated and liquid is not delivered between the measuring tank and culture vessel). However, oxygen and pH can also be controlled in addition to controlling the temperature.

As mentioned above, operating conditions of the operation mode (speed, cycle, angle, etc. of oscillation; time for executing the mode; etc.) may, for example, be selected by a user from those set in the device in advance, or may be set by a user on a case-by-case basis (the same applies to the operating conditions for each of the following operation modes).

If it is necessary to detach adhered cells from the carrier after the pre-culturing step, for example, a detachment method using electromagnetic stimulation may be used in addition to using cell detachment agents such as trypsin or chelating agents.

As shown in FIG. 2, oscillation is performed so that the oscillating plate 102 is tilted only in one direction with respect to a horizontal state. This is for the purposes of preventing the angle of the oscillating plate 102 during the oscillating motion from changing across a horizontal plane, as mentioned above.

In other words, when the oscillating plate 102 is raised counterclockwise with respect to a horizontal plane H as shown in the left side of FIG. 2, the oscillating plate 102 is not tilted more than horizontal (right side FIG. 2) when operated to lie clockwise in the subsequent “oscillating motion” (center in FIG. 2). Tilt angle of oscillation is preferably in the range between 0° and 45° with respect to the horizontal plane H. However, it is not essential to make it horizontal, for example, the oscillation may be performed in the range between 5° and 40°. To obtain a certain degree of agitation effect, it is preferable to make the angle close to horizontal when laid down, and the angle when laid down is preferably 10° or less with respect to the horizontal plane H. Similarly, to obtain a certain degree of agitation effect, it is preferable to make the angle 30° or more with respect to the horizontal plane H when raised.

When the oscillation angle of the oscillating plate 102 is changed across the horizontal plane, the culture fluid CF and the carriers C inside are moved significantly to the left and right as understood from FIG. 2, and physical stress is applied to cells, etc. due to collision of each carrier.

In contrast, as can be seen from the left side and the center of FIG. 2, by tilting the oscillating plate 102 only in one direction with respect to the horizontal state, the movement of the carriers C can be suppressed while moving the culture fluid CF to some extent. In other words, an excellent functional effect can be obtained in reducing physical stress on the cells while agitating the culture fluid CF to some extent. In this embodiment, by using a non-woven fabric strip of carriers (fabric carriers) as the carriers C, movement of the carriers C can be effectively suppressed. In other words, the use of the non-woven fabric strip of carriers C makes the carriers C to be in surface contact with the culture vessel 2, and the carriers C to be in surface contact with each other, exhibiting an effect of making it difficult to move within the culture vessel 2. Examples of the non-woven fabric of carriers applicable to this embodiment include rectangular carriers of about 1 cm square and circular carriers of about 1 cm in diameter. However, the size, quantity, material, surface treatment, etc. are preferably selected properly depending on the application. The rotation speed of the oscillating plate 102 is also preferably adjusted so that the carriers C do not move significantly.

Upon completion of the pre-culturing step, the process moves to the main culturing step (Step 303).

In the operation mode in the main culturing step in Step 303, a feedback control is performed which is to increase the amount of agitation (oscillation) of the culture fluid, etc. compared to the initial culturing, and to operate the pump 112 to deliver the liquid between the measuring tank 109 and the culture vessel 2, while maintaining the oxygen amount and pH of the culture fluid, etc. at predetermined values based on the measurements of the sensor 110 in the measuring tank 109. In the main culturing step, for example, an operation mode in which the motion of oscillation is about once per minute is performed for about 3 days.

The oscillating motion of the oscillating plate 102 in the culturing step is to “tilt only in one direction with respect to a horizontal state” as explained above. Accordingly, physical stress on the cells can be reduced while agitating the culture fluid as mentioned above.

When a transition period to the transfection step arrives, setting is made for the transfection step (Step 304). Transition to the transfection step is preferably made before the cell culture becomes saturated.

In this setting, the culture vessel 2 containing the transfection reagent and the carrier C having adhesive cells attached thereto in the culturing step are installed (with various connections) to the oscillating-type culture device 1, and the liquid in the measuring tank 109 is replaced by the transfection reagent.

Once the setting for transfection is done, the transfection step is performed by transfecting while oscillating on the oscillating plate (Step 305). Transfection is a process of artificially introducing nucleic acids (DNA or RNA) into cells by methods other than viral infection. In this embodiment, the introduction is performed using chemical methods. However, the transfection reagents are not limited to those used in the present invention.

In the transfection mode in Step 305, feedback control is performed which is to operate the pump 112 to deliver the liquid between the measuring tank 109 and the culture vessel 2, while maintaining the oxygen amount and pH of the liquid (transfection reagent) in the culture vessel at predetermined values based on the measurements of the sensor 110 in the measuring tank 109. In the transfection mode, for example, an operation mode in which the motion of oscillation is about once per minute is performed for about 10 days.

The oscillating motion of the oscillating plate 102 in the transfection step is also to “tilt only in one direction with respect to a horizontal state” as explained above. The transfection reagent is also very sensitive to physical stress; however, according to this embodiment, physical stress on the transfection reagent can also be reduced.

As described above, according to the cell production method of this embodiment, since the oscillating plate is tilted only in one direction with respect to a horizontal state, physical stress on cells and transfection reagents can be reduced, making it possible to improve cell culture efficiency and transfection efficiency.

In moving a part of the culture fluid from the culture vessel to the measuring tank to measure dissolved oxygen or pH of the culture fluid, etc. in the measuring tank, delivery of the culture fluid, etc. between the culture vessel and the measuring tank is carried out by controlling the internal pressure of the measuring tank, so as to reduce physical stress on cells and transfection reagent. This makes it possible to improve cell culture efficiency and transfection efficiency.

Regarding “tilting the oscillating plate only in one direction with respect to a horizontal state”, the oscillating angle may be mechanically limited, or the oscillating angle may be limited by control. In other words, the tilt range of the oscillating plate may be mechanically restricted by installing a stopper to limit the tilt angle; or the tilt range of the oscillating plate, which can oscillate across the horizontal plane as a mechanical structure, may be restricted by a control using a control unit or the like.

In this embodiment, the case where the measuring tank is provided and the culture fluid, etc. is moved from the culture vessel to the measuring tank to measure dissolved oxygen and pH in the measuring tank is given as an example. However, the present invention is not limited thereto. As mentioned above, in the case of a flexible culture bag (especially a small one), employing this embodiment is extremely effective. However, in the case where there is no particular hindrance to provide a sensor in the culture vessel, such as when the culture bag is relatively large or the culture vessel is stable, a sensor may be provided in the culture vessel (without a measuring tank).

### Embodiment 2

Embodiment 2 describes a culture vessel used for the cell production method, etc. of Embodiment 1 (which can of course be used for methods and devices other than those of Embodiment 1).

Patent Document 3 discloses a single-use culture vessel (bioreactor bag) that can reduce the effort required for scale-up. The culture vessel disclosed in Patent Document 3 is a rectangular parallelepiped vessel made of a bottom sheet, a top sheet, and sidewall sheets consisting of four surfaces joined together. In the manufacture of such a three-dimensional vessel (joining of each sheet), formation of a part where three sheets are joined is inevitably created near the corners, and welding defects are likely to occur at the parts where the three sheets are joined (welded).

On the other hand, in the case of flat bag-type culture bags (flat culture bags formed by joining two sheets) as described in Patent Documents 3 and 4, which are conventionally used as disposable flexible bags, there is no problem in forming a three-dimensional flexible vessel as described above, since basically only four sides of two flat sheets need to be joined. However, in the case of a relatively small-capacity vessel, the flat culture bag joined on four sides has the problem of difficulty in inflating the bag during use (i.e., difficulty in obtaining a height dimension, and thus, difficulty in making a three-dimensional vessel).

In the culture method of oscillating a culture vessel, it is necessary to allow gas such as oxygen to exist in the culture vessel above the culture fluid and to allow the culture fluid to absorb oxygen, etc. (supply oxygen, etc. to the culture fluid), and therefore, the culture vessel (culture bag) needs to have a height dimension sufficient to form a liquid phase and a gas phase with a predetermined volume (needs to be a three-dimensional vessel).

While a flat culture bag is formed of a flexible sheet, the sheet is configured from a multilayer film to obtain functions required as a culture bag as disclosed in Patent Document 5, and is less elastic. While such a flat culture bag having two less-elastic flat sheets stacked and joined at four sides can be relatively easily inflated (can obtain height dimension) when the bag has a certain size, as the vessel is reduced in capacity (i.e., a smaller bag is made), it becomes more difficult to inflate the bag (i.e., more difficult to obtain the height dimension), making it difficult or impossible to use.

Small-capacity culture vessels suitable for the initial stage of culture and for test culture to determine the conditions of culture, etc., and for such small-capacity culture vessels, there is a need for disposable culture vessels that are easy to use.

A culture vessel 200 of this embodiment is a culture vessel that is easy to use, even for relatively small capacity.

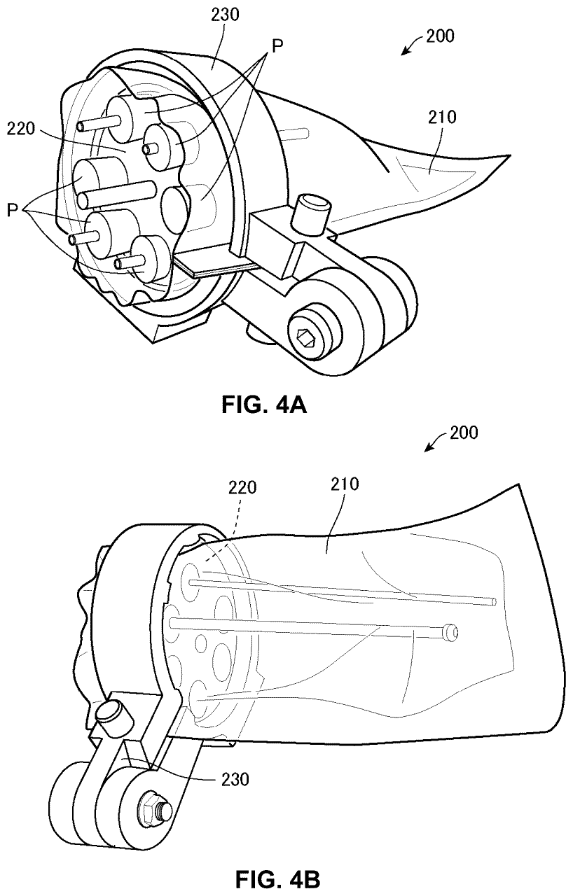

FIG. 4 shows a culture vessel of Embodiment 2 according to the present invention, and FIGS. 4(a) and 4(b) are perspective views seen from different angles, respectively.

The culture vessel 200 of this embodiment is a single-use culture vessel used for cell culture in an oscillating-type culture device, including:
a bag portion 210 formed by joining end regions of opposing sheets leaving an opening,
a plate member 220 having a port member P and occluding the opening, and
a fastening member 230 for closely attaching the plate member 220 to the opening.

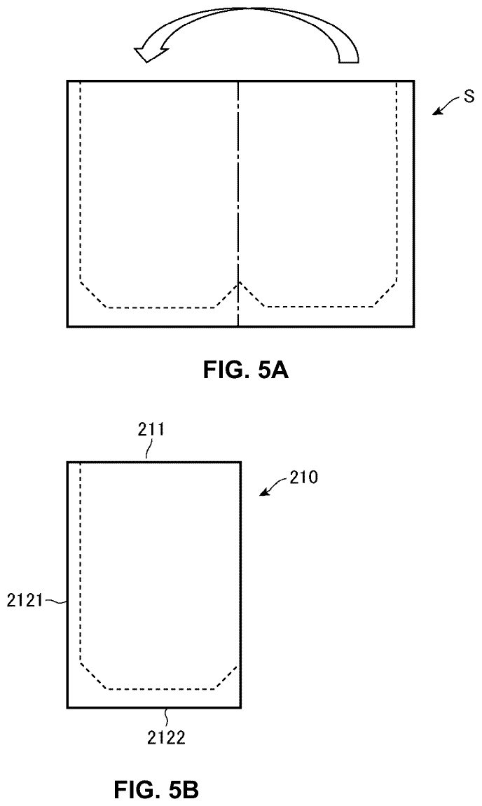

FIG. 5 is an explanatory view of the bag portion 210, in which FIG. 5(a) is a developed view of the bag portion 210, and FIG. 5(b) is a plan view of the bag portion 210.

In the bag portion 210 of this embodiment, “opposing sheets” are formed by folding a single sheet as can be understood from FIG. 5(a), and the end regions of the folded sheet S are joined (welded) leaving the opening 211 as shown in FIG. 5(b), in other words, welded at a side joint margin 2121 and a bottom joint margin 2122 indicated by dotted lines in FIG. 5(b), to thereby form a flat bag shape.

As is clear from the explanation, the flat bag portion 210 can be formed very simply and at low cost.

The sheet S is configured from any material used as a suitable material for use as a culture bag, which is configured from, for example, a single or multi-layered synthetic resin film to have flexibility and impermeability, and a predetermined strength.

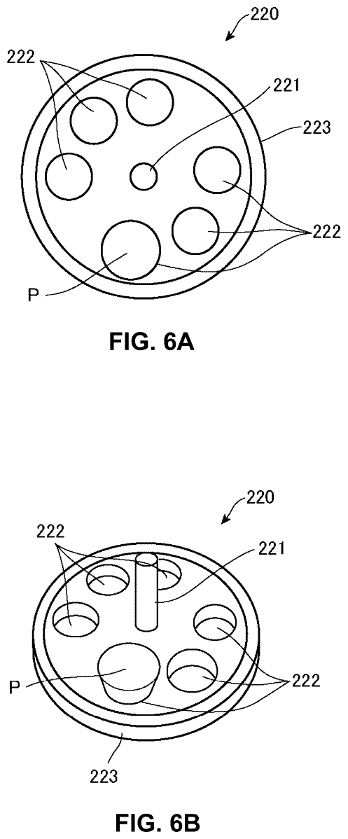

FIG. 6 shows the plate member 220, in which FIG. 6(a) is a plan view, and FIG. 6(b) is a perspective view.

The plate member 220 of this embodiment is a plate-shaped member made of resin that is circular in basic form and has a plurality of port attaching holes 222 for attaching the port member P.

A gripping rod 221 is provided at the center of the plate member 220 to enhance workability in placing the plate member 220 in the opening 211 of the bag portion 210 and fastening with the fastening member 230.

A sealing member 223 is provided on the outer periphery of the plate member 220 to enhance close attachment of the bag portion 210 with the opening 211. The sealing member 223 of this embodiment is a ring-shaped silicone member fitted on the outer periphery of the plate member 220. The outer peripheral dimension of the plate member 220 and the inner peripheral dimension of the opening 211 of the bag portion 210 are preferably about the same or the outer periphery the plate member 220 is preferably slightly smaller. If the outer periphery of the plate member 220 is too small with respect to the inner periphery of the opening 211, problems such as reduced airtightness due to wrinkling may occur. However, according to the culture vessel 200 of this embodiment, since the plate member 220 has the sealing member 223 on its outer periphery and a fastening force can be obtained by the fastening member 230 described later, a close fastening can be made even if the inner periphery of the opening portion 211 of the bag portion 210 is somewhat larger than the outer periphery of the plate member 220.

The port member P attached to the port attaching hole 222 works as a connection for connecting a pipe, etc. for supplying/discharging liquid or gas to/from the culture vessel 200, an attaching portion for attaching various sensors for measuring dissolved oxygen concentration, pH, temperature, etc. of the culture fluid in the culture vessel 200, a sampling port for taking samples from inside the culture vessel 200, etc.

The plate member 220 preferably has a certain thickness from the viewpoint of enhancing close attachment of the bag portion 210 to the opening 211. By having a predetermined thickness, the plate member 220 can have a predetermined strength (rigidity). Since the port member P is attached to the member with a certain rigidity, pipes for supplying/discharging gas, various sensors, etc., which are connected to the port member P, can be attached stably. In conventional pillow-type culture bags, ports are provided on flexible bags (sheets), which can cause problems of pipes for supplying/discharging gas and various sensors, etc. being unstable. However, the culture vessel 200 in this embodiment reduces such problems.

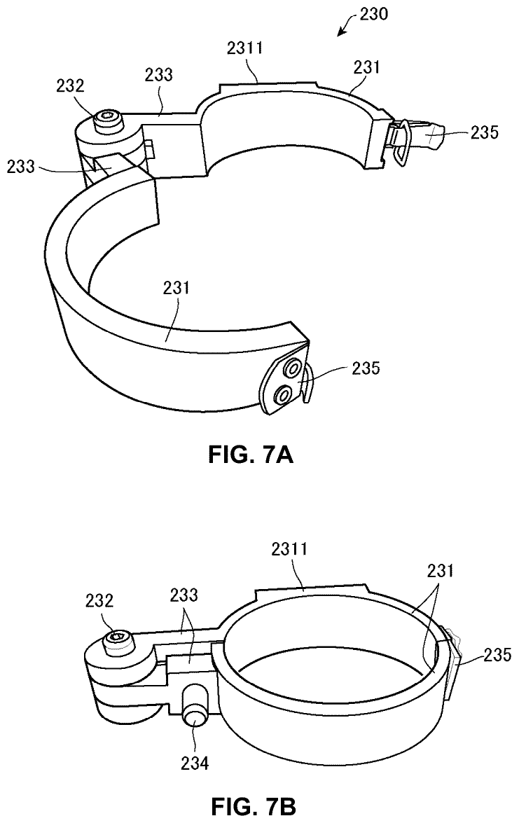

FIG. 7 shows the fastening member 230, in which FIG. 7(a) is a perspective view with the fastening member 230 open, and FIG. 7(b) is a perspective view with the fastening member 230 closed.

The fastening member 230 of this embodiment includes:
two semi-circular members 231 that are substantially circular by being connected to each other,
a flange portion 233 formed on one end of the semi-circular members 231,
a hinge portion 232 pivotally connecting the flange portion 233 provided on each of the two semi-circular members,
a proximal fastening force applying member 234 provided on the flange portion 233, and
a distal fastening force applying member 235 provided on the other end of the semi-circular members 231.

The inner diameter of the semi-circular member 231 is substantially the same as the outer diameter of the plate member 220, or slightly larger than the outer diameter of the plate member 220. The semi-circular members 231 are formed so that, by having both or either of the two semi-circular members 231 formed smaller than half a circle (with a central angle less than 180 degrees), the inner periphery of the two semi-circular members 231 is slightly smaller than the outer periphery of the plate member 220 when the two semi-circular members 231 are put together. In other words, when the two semi-circular members 231 are arranged to contact the outer periphery of the plate member 220, a gap is formed between the two semi-circular members 231 (or the two flange portions 233).

By tightening (urging to reduce or eliminate the gap) the two semi-circular members 231 in this “state with a gap” by the proximal fastening force applying member 234 and the distal fastening force applying member 235, close attachment between the opening 211 of the bag portion 210 and the plate member 220 is obtained.

The hinge portion 232 connects the two semi-circular members 231 having the flange portions 233 to pivot one another as shown in FIGS. 7(a) and 7(b). In the case of a configuration in which the semi-circular members 231 are fastened at both ends by the proximal fastening force applying member 234 and the distal fastening force applying member 235 as in this embodiment, the hinge portion 232 is not an essential configuration. However, each member being connected by the hinge portion 232 in a mutually pivotable manner enhances workability in assembling the culture vessel 200.

The flange portion 233 serves as a connection as the hinge portion 232, and also functions to sandwich the side joint margin 2121 (joined portion of the sheet) of the bag portion 210 as will be explained later.

The proximal fastening force applying member 234 and the distal fastening force applying member 235 are members to obtain fastening force by the fastening member 230. In this embodiment, the proximal fastening force applying member 234 is configured from a screw member that is inserted into the two flange portions 233 (fastening force generates by tightening the screw), and the distal fastening force applying member 235 is configured from a fastener to obtain fastening force through elastic force. For both the proximal fastening force applying member 234 and the distal fastening force applying member 235, any metal fitting or member that enables circumferential fastening of a ring-shaped member can be used. Although two fastening members, the proximal fastening force applying member 234 and the distal fastening force applying member 235, are used herein as an example, one fastening member or three or more fastening members may be used.

In this embodiment, a flat portion 2311 is formed on an outer peripheral surface of one of the two semi-circular members 231. The flat portion 2311 is for stabilizing the culture vessel 200 in placing (placing to lay down) the culture vessel 200. In this embodiment, the flat portion 2311 is formed on only one semi-circular member. However, the flat portion 2311 may be formed on the outer peripheral surfaces of both semi-circular members. The flat portion 2311 (or any member configuring the culture vessel 200, such as the fastening member 230) may be provided with an attachment structure (attachable portion) with respect to the oscillating plate in the oscillating-type culture device. For example, the flat portion 2311 may be provided with a magnet for attachment to the oscillating plate (configuring the oscillating plate 102 from a magnetic material), or it may be removably fixed by members that fit into each other, screws, or other members.

Next, an outline of assembling the culture vessel 200 is explained.

The opening 211 (FIG. 5(b)) of the bag portion 210 formed by a flat bag is spread, and the plate member 220 is placed inside the opening 211. In this process, workability is improved by the presence of the gripping rod 221 on the plate member 220.

With the plate member 220 placed in the opening 211, the fastening member 230 is attached outside at a position that is substantially the same height as the plate member 220. In this situation, it is preferable to position the plate member 220 and the fastening member 230 not to tilt with respect to the bag portion 210, and the presence of the gripping rod 221 improves workability in fine adjustment of the position and angle of the plate member 220 in this situation. The gripping rod 221 may be attachable/removable to/from the plate member 220 (e.g., the gripping rod 221 is attachable/removable (e.g., screwable) to/from the attaching portion (screw hole, etc.) formed in the plate member 220), and the gripping rod 221 may be removed after assembling.

In attaching the fastening member 230, the side joint margin 2121 (joined portion of the sheet) of the bag portion 210 is sandwiched between the two flange portions 233.

This configuration can prevent the likelihood of a gap being formed when the joint margin is folded back and sandwiched between the semi-circular members 231 and the bag portion 210. An excess portion of the bag portion 210 caused by the inner periphery of the bag portion 210 being larger than the outer periphery of the plate member 220 is sandwiched between the two flange portions 233 (the joint margin is pulled and allowed to enter deeper into the flange portions 233), so as to smooth out the loose portion caused by the larger inner periphery of the bag portion 210. This allows the opening 211 of the bag portion 210 to be perfectly aligned with the outer periphery of the plate member 220, and prevents the likelihood of a gap being formed by the wrinkles caused by the loose portion.

Once the above positioning is achieved, the proximal fastening force applying member 234 and the distal fastening force applying member 235 are tightened to closely attach the plate member 220 to the opening 211, thereby configuring the culture vessel 200.

As described above, according to the culture vessel 200 of this embodiment, a three-dimensional vessel can be formed by using the plate member 220 while using the flat bag portion 210 that can be formed very simply and at low cost. As mentioned above, in a conventional, flat culture bag having two less elastic flat sheets stacked and joined at four sides, as the vessel is reduced in capacity, it becomes more difficult to inflate the bag, making it difficult or impossible to use. In contrast, according to the culture vessel 200 of this embodiment, a three-dimensional vessel can be formed while using the flat bag portion 210, thus providing a culture vessel with significantly improved usability even for relatively small-capacity culture bags.

In this embodiment, the case where the plate member 220 is joined to the opening 211 by the fastening member 230 was given an example. However, the present invention is not limited thereto, and any configuration that enables the plate member 220 to be joined to the opening 211 may be adopted. For example, the plate member 220 may be glued or welded to the opening 211, or may be fastened with a member such as a cable tie.

In this embodiment, the fastening member 230 having two semi-circular members was given as an example. However, the fastening member 230 can be configured from a circular member divided into three or more parts.

In using the fastening member, multiple fastening members may be used.

In this embodiment, the bag portion 210 formed by folding a single sheet was given an example. However, the invention is not limited thereto, and can be formed by joining two stacked sheets, etc.

In this case, joint margins would be formed on both sides of the bag. Correspondingly, the number of flange portions can be increased. In other words, the flange portions that were formed only on one end of the two semi-circular members in this embodiment may be formed on both ends of the two semi-circular members.

This may allow the joint margins formed on both sides of the bag to be sandwiched by the flange portions formed on both ends of the semi-circular members, respectively.

In this embodiment, a circular plate member was given as an example. However, the invention is not limited thereto, and may be oval or rectangular (or polygonal) in shape, for example. In using the fastening member, it is obvious that the fastening member should be shaped according to the shape of the plate member.

### REFERENCE SINGS LIST

1 . . . oscillating-type culture device
102 . . . oscillating plate
103 . . . oscillating motion unit
109 . . . measuring tank
110 . . . sensor
112 . . . pump
2, 200 . . . culture vessel
210 . . . bag portion
211 . . . opening
220 . . . plate member
223 . . . sealing member
230 . . . fastening member
231 . . . semi-circular members
232 . . . hinge portion
233 . . . flange portion
234 . . . proximal fastening force applying member
235 . . . distal fastening force applying member
C . . . carrier
P . . . port member

1. A cell production method wherein, in a culturing step for culturing cells while oscillating a culture vessel containing at least cells, a culture fluid, and a carrier on an oscillating plate, tilting the oscillating plate only in one direction with respect to a horizontal state.

2. The cell production method according to claim 1, wherein the culturing step comprises a pre-culturing step and a main culturing step.

3. The cell production method according to claim 1, comprising a transfection step wherein, after the culturing step, a transfection is performed while oscillating the culture vessel containing at least a transfection reagent and the carrier having cells attached thereto on the oscillating plate, such that the oscillating plate is tilted only in one direction with respect to a horizontal state.

4. The cell production method according to claim 1, wherein a tilt angle of the oscillating plate during oscillation is within a range between 0° and 45° with respect to a horizontal plane.

5. The cell production method according to claim 1, wherein a part of the culture fluid is moved from the culture vessel to a measuring tank to measure dissolved oxygen or pH of the culture fluid in the measuring tank, and delivery of the culture fluid between the culture vessel and the measuring tank is carried out by controlling an internal pressure of the measuring tank.

6. The culture vessel used in the cell production method according to claim 1, comprising:
a bag portion formed by joining end regions of opposing sheets leaving an opening; and
a plate member having a port member and occluding the opening.

7. The culture vessel according to claim 6, comprising a fastening member for closely attaching the plate member to the opening.

8. The culture vessel according to claim 7, wherein the plate member is circular, and wherein the fastening member comprises:
two semi-circular members that are substantially circular by being connected to each other;
a flange portion formed on one end of the semi-circular members; and
a fastening force applying member that generates a fastening force for closely attaching the plate member to the opening.

9. The culture vessel according to claim 8, wherein the opposing sheets are formed by folding a single sheet, and a joined portion of a folded sheet is sandwiched by the flange portions.

10. The culture vessel according to claim 6, comprising a sealing member on an outer periphery of the plate member.

11. The culture vessel according to claim 6, further comprising an attachable portion for attachment to the oscillating plate.

12. An oscillating-type culture device used in the cell production method according to claim 1, comprising:
the oscillating plate;
an oscillation control unit for controlling oscillation of the oscillating plate for tilting the oscillating plate only in one direction with respect to a horizontal state;
a sensor value acquisition unit for acquiring a sensor value from a sensor attached to the culture vessel; and
a gas supply control unit for supplying oxygen or carbon dioxide into the culture vessel based on the sensor value.

13. A culture vessel for cell culture, comprising:
a bag portion formed by joining end regions of opposing sheets leaving an opening; and
a plate member having a port member and occluding the opening.
